(19) United States
(12) Patent Application Publication
(10) Pub. No.: US 2018/0174486 A1
(43) Pub. Date: Jun. 21, 2018

(54) IMPAIRMENT DETECTION DEVICE WITH PERFORMANCE FEEDBACK SYSTEM AND METHOD OF OPERATING THE SAME

(71) Applicant: GM GLOBAL TECHNOLOGY OPERATIONS LLC, Detroit, MI (US)

(72) Inventors: FRED W. HUNTZICKER, ANN ARBOR, MI (US); WILLIAM A. BIONDO, BEVERY HILLS, MI (US); DAVID T. PROEFKE, TROY, MI (US)

(73) Assignee: GM GLOBAL TECHNOLOGY OPERATIONS LLC, Detroit, MI (US)

(21) Appl. No.: 15/386,606

(22) Filed: Dec. 21, 2016

(51) Int. Cl. G09B 19/24; G09B 5/02; G09B 5/04; G09B 5/00; A61B 5/18; G01N 33/00; G01N 33/497

(52) U.S. Cl. CPC G09B 19/24; G09B 5/02; G09B 5/04; G09B 5/00; G01N 2033/4975; G01N 33/0073; G01N 33/4972; G01N 33/007; A61B 5/18

(57) ABSTRACT

Methods and apparatus are provided for an impairment detection device. The apparatus comprises a feedback device that provides feedback during an impairment test. The feedback is configured to inform the user how to change their behavior to satisfy predetermined conditions during the test.

### INTRODUCTION

The present disclosure generally relates to impairment detection devices and, more particularly, relates to impairment detection devices with performance feedback systems and methods of operating the same.

Various impairment detection devices are used to detect whether a person is impaired by a certain substance (e.g., alcohol, tetrahydrocannabinol (THC), cocaine, etc.). For example, breath analysis devices such as alcohol breathalyzers estimate the amount of the impairing substance within the user's blood. The test subject or user blows into the device, and a sensor estimates the blood-alcohol content of the user from the breath sample.

In many cases, being tested by these devices can be inconvenient and frustrating. The user may have difficulty following the testing procedures. As a result, it may be difficult to complete the test and/or obtain accurate test results. When using a breath analysis device, for example, the user might inadvertently blow too hard into the device, thereby reducing the device's ability to obtain a reading from the breath sample. Conversely, the sensor may not be able to complete an accurate analysis if the test subject blows too lightly into the device. Also, the test subject may run out of air before the sensor can provide an accurate reading.

Accordingly, it is desirable to provide an impairment detection device that improves the testing process for the user. More specifically, it is desirable to provide a device that provides feedback informing the user how to adjust their testing behavior so that the device can gather accurate test results. Furthermore, other desirable features and characteristics of the present disclosure will become apparent from the subsequent detailed description and the appended claims, taken in conjunction with the accompanying drawings and the background of the present disclosure.

### SUMMARY

An impairment detection device is provided for performing an impairment analysis on a user. The impairment detection device comprises an input device configured to receive a user input during a testing phase of the impairment analysis. The impairment detection device also includes a first sensor configured to analyze the user input to observe an amount of an impairing substance of the user. Furthermore, the impairment detection device includes a second sensor configured to observe a current performance parameter associated with the input, the current performance parameter being changeable by the user during the testing phase. Also, the impairment detection device includes a feedback device configured to provide feedback to the user during the testing phase. Additionally, the impairment detection device includes a controller with a processor configured to compare the current performance parameter to a predetermined target parameter and determine a difference between the current performance parameter and the predetermined target parameter. The controller is configured to control the feedback device to provide feedback during the testing phase. The feedback is configured to inform the user how to change the current performance parameter to reduce the difference between the current performance parameter and the predetermined target parameter.

A method is provided for operating an impairment detection device having an input device, a controller with a processor, a first sensor, a second sensor, and a feedback system. The method comprises analyzing, with the first sensor, a user input provided during a testing phase to the input device. The method also comprises observing, with the first sensor, an amount of an impairing substance of the user as a result of the analysis of the user input. Moreover, the method includes observing, with the second sensor, a current performance parameter associated with the user input. The current performance parameter is changeable by the user during the testing phase. The method additionally includes comparing, with the processor, the current performance parameter to a predetermined target parameter to obtain a difference between the current performance parameter and the predetermined target parameter. Furthermore, the method includes providing, with the feedback system, feedback configured to inform the user how to change the current performance parameter during the testing phase to reduce the difference between the current performance parameter and the predetermined target parameter.

### BRIEF DESCRIPTION OF THE DRAWINGS

The present disclosure will hereinafter be described in conjunction with the following drawing figures, wherein like numerals denote like elements, and:

FIG. 1A is a schematic diagram of an impairment detection device according to exemplary embodiments of the present disclosure;

FIG. 1B is a perspective view of an input device of the impairment detection device according to exemplary embodiments of the present disclosure;

FIG. 2 is a data flow diagram of the impairment detection device of FIG. 1A;

FIG. 3 is a flowchart illustrating a method of operating the impairment detection device of FIG. 1A; and

FIG. 4 is a schematic diagram of the impairment detection device according to additional embodiments of the present disclosure.

### DETAILED DESCRIPTION

The following detailed description is merely exemplary in nature and is not intended to limit the present disclosure or the application and its uses. Furthermore, there is no intention to be bound by any theory presented in the preceding background or the following detailed description.

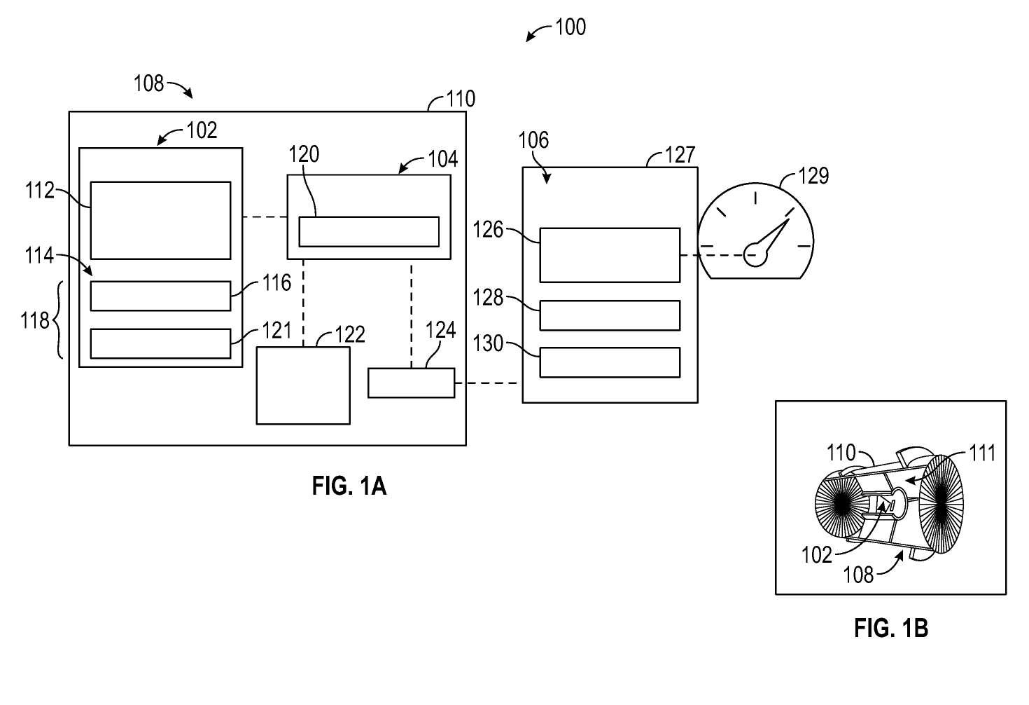

Referring to FIG. 1A, an impairment detection device 100 is illustrated according to exemplary embodiments of the present disclosure. As will be discussed, the impairment detection device 100 provides feedback during testing. The feedback informs the user how to adjust their behavior so that the device 100 can accurately perform the test.

In some embodiments, the device 100 detects a performance parameter of the user. The device 100 may compare the detected performance parameter to at least one predetermined parameter. Also, the device 100 may determine whether the detected performance parameter is above or below the predetermined parameter. The device 100 provides feedback based on this comparison and determination. This feedback informs the user whether to increase or decrease the performance parameter so that the current performance parameter approaches the predetermined parameter. Thus, the device 100 is configured to provide feedback (i.e., performance feedback) informing the user how to change their behavior so that the device 100 can complete an accurate analysis. In a sense, the device 100 is configured to train the test subject how to behave during the test so that the testing can be completed quickly and accurately.

For example, in the case of a breath analyzer (i.e., breathalyzer, breath analysis device), the device 100 may observe how hard the user is blowing into the device 100. In other words, the device 100 may observe the flow rate of the breath being delivered to the device 100 from the user. The device 100 may compare the observed flow rate to a predetermined target flow rate. Based on this comparison, the device 100 provides feedback informing the user whether to blow harder (i.e., increase flow rate) or whether to blow softer (i.e., decrease flow rate). In some embodiments, the device 100 may provide continuous and updated feedback (i.e., dynamic feedback) during the test and until the test is completed. Accordingly, the user is more likely to blow air into the device 100 at the flow rate needed for performing an accurate test.

Other impairment detection devices also fall within the scope of the present disclosure, such as a touch-based (i.e., tactile) impairment detection device. The user may touch and apply pressure to a specified area of the device. During the impairment test, the device may direct a light (e.g., a laser) at the user's skin and observe the resulting light signal to detect the amount of an impairing substance within the user's body. During the test, the device may detect the amount of pressure applied by the user and provide feedback indicating whether to apply more pressure or whether to apply less pressure to the device. Other impairment detection devices also fall within the scope of the present disclosure as well.

Moreover, it will be appreciated that the impairment detection device 100 may be configured for detecting one or more of a variety of impairing substances. The device 100 may be configured for detecting alcohol (e.g., ethanol) in some embodiments. In additional embodiments, the device 100 may be configured for detecting tetrahydrocannabinol (THC), the psychoactive compound found in marijuana. In further embodiments, the device 100 may be configured for detecting cocaine or other impairing substance.

One or more components of the device 100 may be embodied with computer-based devices. Embodiments of the present disclosure may be described herein in terms of functional and/or logical block components and various processing steps. It should be appreciated that such block components may be realized by any number of hardware, software, and/or firmware components configured to perform the specified functions. For example, an embodiment of the present disclosure may employ various integrated circuit components, e.g., memory elements, digital signal processing elements, logic elements, look-up tables, or the like, which may carry out a variety of functions under the control of one or more microprocessors or other control devices. In addition, those skilled in the art will appreciate that embodiments of the present disclosure may be practiced in conjunction with any number of systems, and that the impairment detection devices described herein are merely exemplary embodiments of the present disclosure.

For the sake of brevity, conventional techniques related to signal processing, data transmission, signaling, control, and other functional aspects of the systems (and the individual operating components of the systems) may not be described in detail herein. Furthermore, the connecting lines shown in the various figures contained herein are intended to represent example functional relationships and/or physical couplings between the various elements. It should be noted that many alternative or additional functional relationships or physical connections may be present in an embodiment of the present disclosure.

As used herein, the term module refers to any hardware, software, firmware, electronic control component, processing logic, and/or processor device, individually or in any combination, including without limitation: application specific integrated circuit (ASIC), an electronic circuit, a processor (shared, dedicated, or group) and memory that executes one or more software or firmware programs, a combinational logic circuit, and/or other suitable components that provide the described functionality.

Referring now to FIGS. 1A and 1B, the impairment detection device 100 will be discussed more specifically according to exemplary embodiments. As shown, the impairment detection device 100 may include an input device 108, a sensor system 102, a controller 104, and a feedback system 106. Each of these components will be discussed in detail below according to exemplary embodiments. For purposes of discussion, the impairment detection device 100 will be initially described as a breath analysis device. Other embodiments will be discussed later in the description.

The input device 108 is shown schematically in FIG. 1A and is shown in a perspective view according to an example embodiment in FIG. 1B. The input device 108 may be configured for receiving an input from the user (i.e., the test subject). The input device 108 may be configured to receive a volume of exhaled air or other type of input from the user. In some embodiments, the input device 108 may include a housing 110. As shown in FIG. 1B, the housing 110 may be a hollow shell that at least partially encloses an interior space 111. In the case of a breath analysis device, for example, the user may blow air into an inlet 113 of the housing 110 during the test. As shown in FIGS. 1A and 1B, the housing 110 may support the sensor system 102 and the controller 104. The feedback system 106 may be remote from the housing 110 as will be discussed. In other embodiments, the housing 110 may support the sensor system 102, the controller 104, and the feedback system 106.

The sensor system 102 may include one or more sensors for observing various conditions and parameters during and/or after the test. As shown in the embodiment of FIG. 1A, for example, the sensor system 102 may include an impairing substance sensor 112 (i.e., a first sensor) that is configured to analyze the user input to observe an amount of impairing substance of the user. In some embodiments, the substance sensor 112 may be an alcohol sensor that observes alcohol content within the breath sample input by the user. In other embodiments, the substance sensor 112 may be a THC sensor that observes THC content within the breath sample. Moreover, in some embodiments, the substance sensor 112 may be a cocaine sensor that observes cocaine content within the breath sample. The sensor 112 may be configured for detecting other impairing substances as well.

The sensor system 102 may additionally include a performance sensor 114 (i.e., a second sensor). The performance sensor 114 may be configured to observe a parameter that is associated with the input to the input device 108. The performance sensor 114 may be configured to observe a parameter that is associated with the user's behavior during delivery of the input. The performance sensor 114 may be configured to detect a current performance parameter as the user delivers, for example, a breath of air to the input device 108. It will be appreciated that the performance parameter observed by the sensor 114 may be changeable by the user during the test.

In some embodiments, the performance sensor 114 may be a gas flow sensor 116. The flow sensor 116 may observe a parameter related to the current flow of breath that the user is blowing into the housing 110 during the testing. For example, the flow sensor 116 may observe a flow rate of the exhaled breath. As an example, the flow sensor 116 may be embodied by a mass flow sensor. Specifically, the flow sensor 116 may be a thermal mass flow meter. In some embodiments, the performance sensor 114 may be a thermal mass flow meter that is commercially available from Universal Flow Monitors, Inc. of Hazel Park, Michigan. The thermal flow sensor 116 may measure the gas mass flow using the thermal properties of the breath. The thermal flow sensor 116 may include a pair of wires or other probes that are exposed to the breath sample being delivered into the housing 110. One of the wires/probes may be considered a heater probe because a measured amount of heat is supplied thereto. Some of this heat is lost to the flowing breath sample. As flow increases, more heat is lost. The amount of heat lost is sensed using temperature measurements gathered by the second wire/probe. The transmitter uses the heat input to the first wire/probe and the temperature measurements obtained by the second wire/probe to determine fluid flow of the breath sample delivered by the user. The flow sensor 116 may be configured to dynamically and continuously observe the mass flow rate of the breath sample over the duration of the test. Thus, the flow sensor 116 may dynamically and continuously observe the mass flow rate as it varies during the duration of the test.

The flow sensor 116 may be a mass flow sensor that is configured differently in other embodiments of the present disclosure. For example, the flow sensor 116 may include a Coriolis flow sensor, an ultrasonic meter, or a rotometer in different embodiments of the present disclosure. Also, in some embodiments, the flow sensor 116 may be a volumetric flow sensor in some embodiments.

Additionally, the sensor system 102 may include a third sensor 118. The third sensor 118 may be configured to observe a quantity of the breath delivered to the input device 108. As will be discussed, data obtained from the third sensor 118 may be used to determine when the test has been completed. With the third sensor 118, the device 100 may be configured to inform the user when a sufficient amount of breath has been blown into the housing 110. Thus, the third sensor 118 may be considered a quantity sensor.

In some embodiments, the third sensor 118 may be collectively embodied by the flow sensor 116 and a timer 121. The flow sensor 116 may be configured as discussed above. The timer 121 may time the duration of the test. It will be appreciated that the amount of breath delivered as the user blows into the housing 110 is a function of the mass flow rate observed by the flow sensor 116. Thus, as will be discussed, data from the flow sensor 116 and the timer 121 may be gathered to determine how much breath (i.e., the volume of breath) the user has blown into the housing 110.

The controller 104 may include a processor 120. The processor 120 of the impairment detection device 100 generally represents the hardware, software, and/or firmware components configured to facilitate communications and/or interaction between the sensor system 102, the feedback system 106, and/or other elements of the device 100. The processor 120 may also perform additional tasks and/or functions described in greater detail below. Depending on the embodiment, the processor 120 may be implemented or realized with a general-purpose processor, a content addressable memory, a digital signal processor, an application specific integrated circuit, a field programmable gate array, any suitable programmable logic device, discrete gate or transistor logic, processing core, discrete hardware components, or any combination thereof, designed to perform the functions described herein. The processor 120 may also be implemented as a combination of computing devices, e.g., a plurality of processing cores, a combination of a digital signal processor and a microprocessor, a plurality of microprocessors, one or more microprocessors in conjunction with a digital signal processor core, or any other such configuration. In practice, the processor 120 includes processing logic that may be configured to carry out the functions, techniques, and processing tasks associated with the operation of the impairment detection device 100. Furthermore, the steps of a method or algorithm described in connection with the embodiments disclosed herein may be embodied directly in hardware, in firmware, in a software module executed by the processor 120, or in any practical combination thereof.

Additionally, the impairment detection device 100 may include a data storage module 122. The data storage module 122 may be realized as RAM memory, flash memory, EPROM memory, EEPROM memory, registers, a hard disk, a removable disk, a CD-ROM, or any other form of storage medium known in the art. In this regard, the data storage module 122 may be coupled to the processor 120 such that the processor 120 may read information from, and write information to, the data storage module 122. In the alternative, the data storage module 122 may be integral to the processor 120. As an example, the processor 120 and the data storage module 122 may reside in an ASIC.

The impairment detection device 100 may further include a communication module 124. The communication module 124 may be configured to provide wired and/or wireless intercommunication between the controller 104 and the feedback system 106.

The feedback system 106 may include one or more of a visual feedback device 126, an audio feedback device 128, and a tactile feedback device 130. As will be discussed, the feedback system 106 may be controlled by the controller 104 to provide feedback to the user during the duration of the test. This feedback can inform and train the user how to provide a useable test input.

In some embodiments, impairment detection device 100 may be configured for use and in association with a vehicle 127, such as a passenger car, a bus, a van, a truck, a watercraft, and/or an aircraft. The impairment detection device 100 may be used as a keylock system. As such, the user provides a satisfactory sample of breath before operating the vehicle 127. Accordingly, the user proves via the impairment detection device 100 that the user has less than a predetermined amount of alcohol, THC, cocaine, or other substance within his or her system.

In some embodiments, the input device 108 may be a hand-held device, whereas the feedback system 106 may be supported by the vehicle. For example, the visual feedback device 126 may be a configurable display of the vehicle. The display may be implemented using any one of numerous known displays suitable for rendering textual, graphic, and/or iconic information in a format viewable by the operator. Non-limiting examples of such displays include various cathode ray tube (CRT) displays, and various flat panel displays such as various types of LCD (liquid crystal display) and TFT (thin film transistor) displays. As represented in FIG. 1A, the visual feedback device 126 may also include a vehicle gauge 129, such as the vehicle speedometer, odometer, or another vehicle gauge. Additionally, the visual feedback device 126 may be a component of the vehicle's entertainment system. Also, the audio feedback device 128 may be part of the audio system of the vehicle and may be mounted within the vehicle console, dashboard, doors, and/or roof liner of the vehicle. Furthermore, the tactile feedback device 130 may be supported within a vehicle seat, within the steering wheel, within an armrest, or another area of the vehicle.

It will be appreciated that the feedback system 106 may provide various types of feedback without departing from the scope of the present disclosure. The visual feedback device 126 can provide alphanumeric messages in conjunction with verbal messages from the audio feedback device 128. The visual feedback device 126 may provide symbols, icons, etc. as well. Moreover, the visual feedback device 126 may be lights or lamps that emit different colors, blinking patterns, etc. to communicate the various messages, alerts, and alarms to the user. Additionally, if the visual feedback device 126 includes a speedometer, odometer, or other gauge 129, then those gauges can be configured as a meter. In some embodiments, a display may inform the user to attempt blowing that maintains the gauge between a predetermined range on the gauge. In a specific embodiment, the display or speaker may output a message to blow hard enough to keep the speedometer between 30 m.p.h. and 60 m.p.h. Furthermore, the audio feedback device 128 may provide verbal alerts and/or symbolic tones in order to communicate the information to the user. Additionally, the tactile feedback device 130 may provide vibrations at different frequencies to distinguish between the different feedback signals delivered to the user.

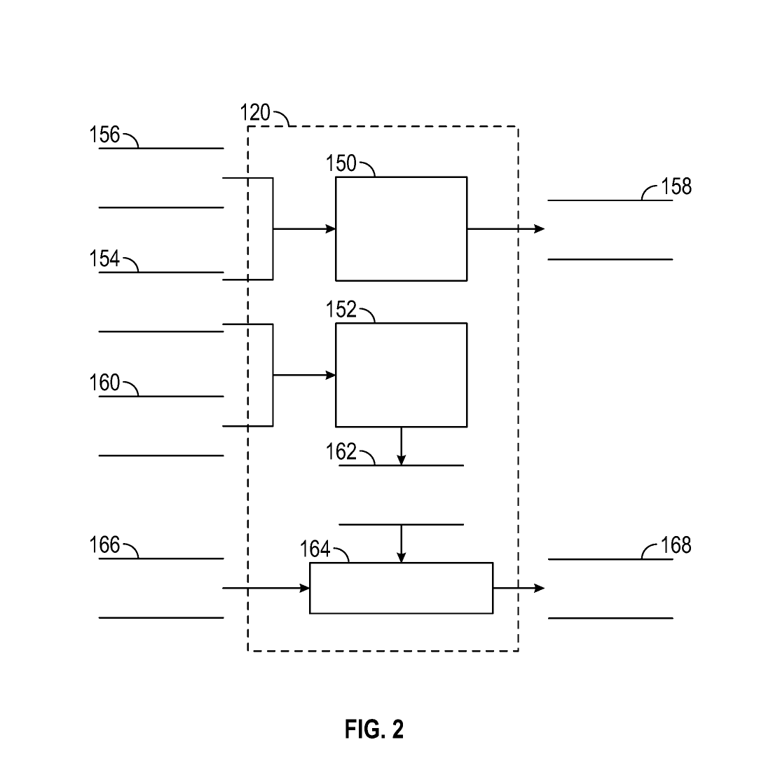

Referring now to both FIGS. 1A and 2, the processor 120 and the dataflow through the processor 120 will be discussed according to exemplary embodiments. The embodiment of FIG. 2 represents embodiments in which the flow of the breath sample into the input device 108 is analyzed. However, it will be appreciated that the data flow of FIG. 2 may be adapted for other types of analyses as well without departing from the scope of the present disclosure.

As shown in FIG. 2, the processor 120 may include a flow comparison module 150. The flow comparison module 150 may be used for controlling feedback specifically intended to train the user how to use the device 100 during the duration of the test. For example, the flow comparison module 150 may be used to control feedback informing the user whether to blow harder (i.e., increase mass flow rate) into the input device 108 and/or whether to blow softer (i.e., decrease mass flow rate) into the input device 108 during the duration of the test.

The flow comparison module 150 may receive target flow data 156 from the data storage module 122. The target flow data 156 may include a predetermined range of mass flow rate limits. For example, the target flow data 156 may include a lower limit flow rate, an upper limit flow rate, and an intermediate target flow rate (i.e., an ideal flow rate) that is between the lower limit and the upper limit. In some embodiments, the intermediate target flow rate included in the target flow data 156 is the flow rate that allows the impairing substance sensor 112 (FIG. 1A) to most effectively analyze the breath sample delivered by the user. For example, the intermediate target flow rate may be the flow rate at which the substance sensor 112 can most quickly analyze the breath sample.

The flow comparison module 150 may also receive current flow data 154 output from the flow sensor 116. Thus, the flow data 154 may represent the current mass flow rate of the breath sample as it is delivered by the user to the input device 108. In other words, the flow sensor 116 may convert the actual flow rate into an electrical signal and send the signal to the flow comparison module 150 of the processor 120.

The flow comparison module 150 may compare the target flow data 156 to the current flow data 154. Accordingly, the flow comparison module 150 may determine the difference between the current flow rate (represented in the current flow data 154) and the one or more predetermined flow rates (represented in the target flow data 156). In some embodiments, the flow comparison module 150 may determine: 1) whether the current flow rate is between the lower limit flow rate and the upper limit flow rate; 2) whether the current flow rate is below the lower limit flow rate; or 3) whether the current flow rate is above the upper limit flow rate. Also, in some embodiments, the flow comparison module 150 may determine whether the current flow rate is approximately equal to the predetermined target flow rate.

Furthermore, in some embodiments, the flow comparison module 150 may determine the difference between the current flow rate (represented in the current flow data 154) and the one or more predetermined flow rates (represented in the target flow data 156).

Based on the comparison of the current flow data 154 and the target flow data 156, the flow comparison module 150 may output flow feedback data 158. The flow feedback data 158 may be control signals that are communicated to the feedback system 106. Generally, if the processor 120 determines that the current flow rate is above the upper limit flow rate, one or more of the feedback devices 126, 128, 130 may inform the user to blow softer into the input device 108. Conversely, if the processor 120 determines that the current flow rate is below the lower limit flow rate, one or more of the feedback devices 126, 128, 130 may inform the user to blow harder into the input device 108. In other words, the feedback may inform the user how to change the current flow rate (represented in the current flow data 154) to reduce the difference between the one or more predetermined flow rates (represented in the target flow data 156). Additionally, if the processor 120 determines that the current flow rate is approximately equal to the target flow rate, one or more of the feedback devices 126, 128, 130 may inform the user to maintain the same blowing behavior.

The flow feedback data 158 may cause the feedback device(s) 126, 128, 130 to output different types of feedback depending on the value of the current flow rate in comparison to the range of predetermined flow rates. In other words, the feedback device(s) 126, 128, 130 may output a first type of feedback when the current flow rate is below the lower limit flow rate, a second type of feedback when the current flow rate is above the upper limit flow rate, and a third type of feedback when the current flow rate is approximately equal to the intermediate target flow rate. For example, the visual feedback device 126 may display a first visual message informing the user to blow harder when the current flow rate is below the lower limit flow rate. In this example, the visual feedback device 126 may display a second visual message informing the user to blow softer when the current flow rate is above the upper limit flow rate. Moreover, the visual feedback device 126 may display a third visual message informing the user to maintain the current blowing behavior when the current flow rate is approximately equal to the intermediate target flow rate.

As shown in FIG. 2, the processor 120 may also include a quantity calculation module 152. The quantity calculation module 152 may receive the current flow data 154 in addition to time data 160. The current flow data 154 is discussed above. The time data 160 may be received from the timer 121. The time data 160 may represent the amount of time that has elapsed from the start of the test. Since the quantity (i.e., volume) of the breath sample delivered to the input device 108 may be a function of time, the quantity calculation module 152 may rely on an algorithm for calculating the amount of delivered breath based on the current flow data 154 and the time data 160. The result of the calculation may be output from the quantity calculation module 152 as current quantity data 162.

The processor 120 may further include a quantity comparison module 164. The quantity comparison module 164 may be used for controlling feedback specifically intended to inform the user when the test has been completed. Generally, the quantity comparison module 164 may be used to detect when the necessary amount of breath has been delivered to the impairing substance sensor 112 (FIG. 1A) to complete the test. As a result, the quantity comparison module 164 may be used to control feedback informing the user that a sufficient amount of breath has been delivered and that testing is complete.

The quantity comparison module 164 may receive the current quantity data 162 discussed above. The quantity comparison module 164 may further receive target quantity data 166. The target quantity data 166 may be received from the data storage module 122 and may represent a predetermined quantity limit (i.e., a volume of breath that is necessary for completing an accurate analysis).

The quantity comparison module 164 may compare the current quantity of delivered breath (represented in the current quantity data 162) to the predetermined quantity limit (represented in the target quantity data 166). Based on this comparison, the quantity comparison module 164 may output quantity feedback data 168. The quantity feedback data 168 may be control signals for controlling one or more of the feedback devices 126, 128, 130. For example, in some embodiments, the quantity feedback data 168 may cause the feedback device(s) 126, 128, 130 to output an alert that the current quantity of delivered breath (represented by current quantity data 162) is at least equal to the predetermined quantity limit (represented by the target quantity data 166). In some embodiments, the feedback device(s) 126, 128, 130 may output both: 1) a first feedback message informing the user that the testing is proceeding and to keep blowing into the input device 108; and 2) a second feedback message informing the user that the testing is complete and that there is no need to continue blowing into the input device 108.

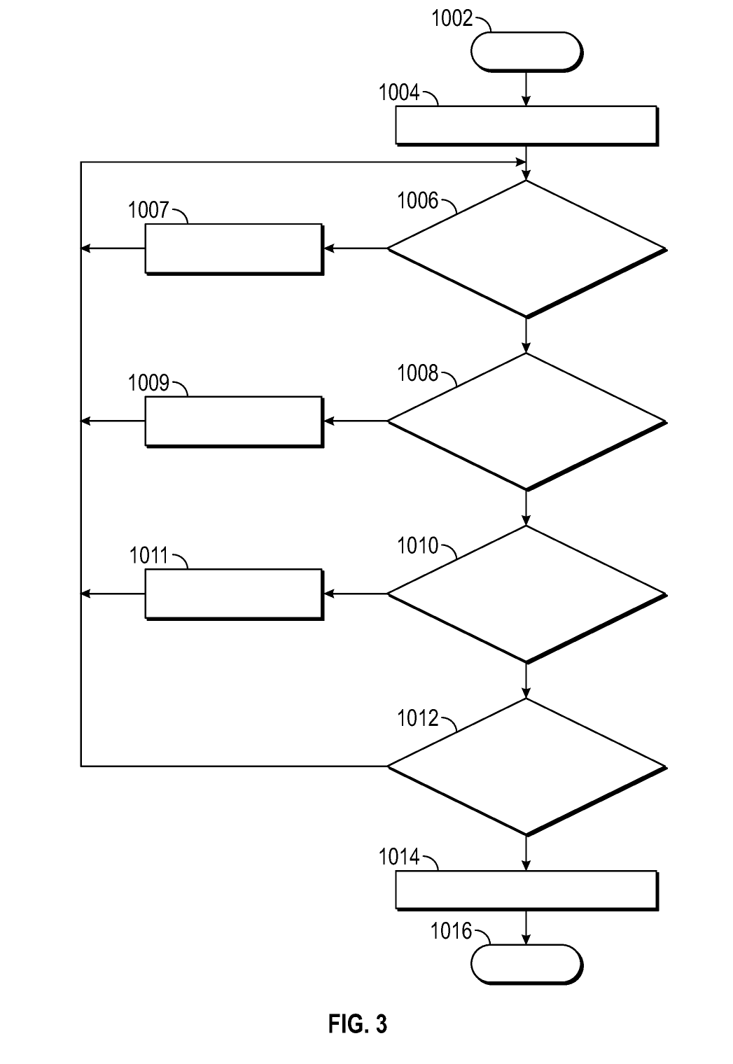

Referring now to FIG. 3, a method 1000 of operating the impairment detection device 100 is illustrated according to exemplary embodiments. The method 1000 may begin at 1002. In some embodiments, 1002 may represent the beginning of a testing phase. In some embodiments, a user may input a user command, for example, by pressing a button on the device 100 to begin the method 1000.

The method 1000 may continue, at 1004, and the feedback system 106 may output a first alert that the test is starting, is about to start, or otherwise. The alert of 1004 may also instruct the user to begin blowing into the input device 108.

Next, at 1006, the processor 120 may determine whether the current flow rate (observed by the flow sensor 116 and associated with the current flow data 154) is greater than the upper limit target flow rate (stored in the data storage device 122 and associated with the target flow data 156). If so, then at 1007, the feedback system 106 may provide corresponding feedback informing the user to blow more lightly (i.e., less turbulently). Subsequently, the method 1000 may loop back to 1006.

If, at 1006, the processor 120 determines that the flow is not above the upper flow rate limit, then the method 1000 may continue to 1008. At 1008, the processor 120 may determine whether the current flow rate is below the lower limit 1008. If so, then at 1009, the feedback system 106 may provide corresponding feedback informing the user to blow harder to deliver the exhalation at a higher flow rate. Subsequently, the method 1000 may loop back to 1006.

Assuming that the flow rate is between the upper limit and the lower limit (negative results at the decisions of 1006 and 1008), then the method 1000 may continue at 1010. At 1010, the processor 120 may determine whether the current flow rate is approximately equal to the intermediate target flow rate. If so, then the method 1000 may continue to 1011, and the feedback system 106 may provide corresponding feedback informing the user to maintain the same flow rate during delivery of the breath sample. Then, the method 1000 may loop back to 1006.

During this test method 1000 and while the user is blowing into the input device 108, the impairing substance sensor 112 may simultaneously analyze the contents of the breath sample. The method 1000 represented in FIG. 3 may continue until the processor 102 determines that the impairing substance sensor 112 can perform an accurate analysis. Specifically, at 1012, the processor 120 may determine whether the quantity of received breath sample (as observed by quantity sensor 118 and associated with the current quantity data 162) is less than the target quantity (stored in the data storage device 122 and associated with the target quantity data 166). If yes, then the method 1000 may loop back to 1006. If not, then the method 1000 may continue to 1014, wherein the feedback system 1016 may provide an alert that the test has been completed.

Accordingly, it will be appreciated that the impairment detection device 100 may provide continuous, dynamic, and updated feedback as the test progresses up to, and including, the completion of the test. This feedback provides the user with valuable information during the test. The user can accurately know how to adjust their behavior so that the test can be completed quickly and accurately. Also, the user can be trained for future testing. Moreover, the results of the impairment detection test (e.g., the alcohol, THC, and/or cocaine content detected by the sensor 112) may be output to the user via the feedback system 106.

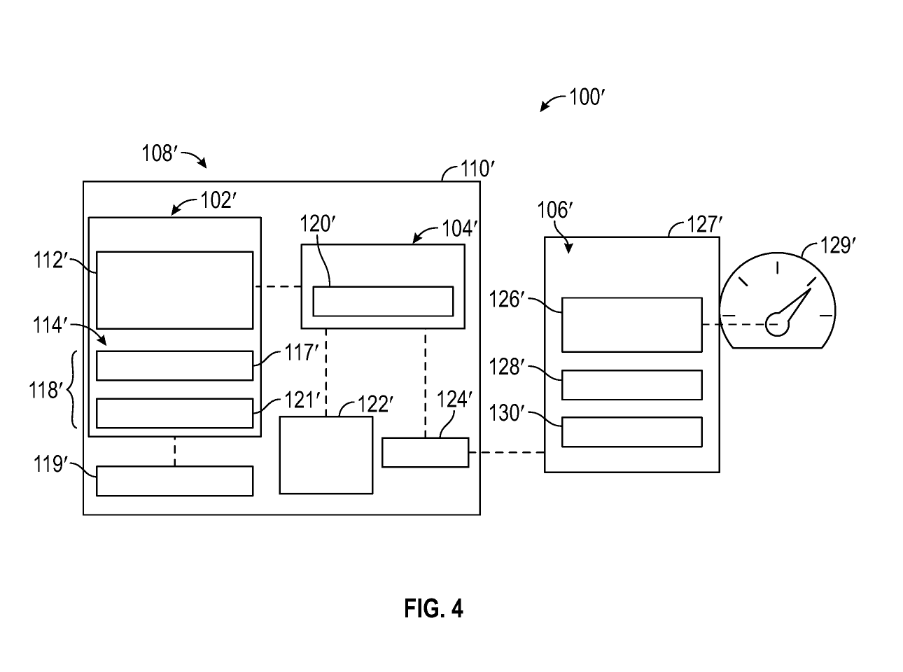

It will be appreciated that the device 100 and its method 1000 of operation may vary from those discussed above. For example, additional embodiments of the impairment detection device 100′ are illustrated in FIG. 4. The device 100′ may have features that are substantially similar to the device 100 illustrated in FIG. 1A except as detailed below. Features that correspond to those of FIG. 1A are identified with corresponding reference numbers.

In the embodiment of FIG. 4, the input device 108′ may be configured to receive a tactile, touch-based, or pressure-based user input. For example, in some embodiments, the user may grasp, touch, and contact a specified area on the input device 108′ to provide the tactile user input.

Also, the device 100′ may include an emitter 119′ configured to emit a light toward the user's body part that is touching the input device 108′. The light may interact with the body part to produce a light response. For example, the light may transmit through and/or reflect from the user's body. The light response may depend on the amount of impairing substances within the user's body. In some embodiments, for example, the light from the emitter 119′may transmit through and/or reflect from capillary blood vessels, and the light response may depend on the blood flow (e.g., blood mass flow rate) through the capillaries. The impairing substance sensor 112′ may detect and observe the light response for estimating and detecting the amount of an impairing substance (e.g., alcohol, THC, cocaine, etc.) within the user's blood.

Additionally, the performance sensor 114′ may include a pressure sensor 117′ that is configured to detect how much pressure the user is applying during the impairment test (i.e., the current pressure associated with the tactile user input) as the impairment substance sensor 112′ is detecting the light response. The processor 120′may compare the current pressure detected by the sensor 112′ to at least one predetermined pressure threshold stored in the data storage device 122′.

Based on this comparison, the feedback system 106′ may provide feedback to the user. The feedback provided may indicate to the user whether to reduce the pressure applied to the input device 108′ during the test and/or increase the applied pressure. The feedback may also inform the user that the current applied pressure matches a preferred pressure. Accordingly, the feedback may inform and train the user to provide tactile input that is suitable for producing an accurate impairment test.

In other words, the processor 120′ may compare the current amount of pressure to one or more predetermined pressures (e.g., a lower limit pressure, an upper limit pressure, and an intermediate pressure). The processor 120′ may determine whether the current amount falls below the lower limit pressure, is above the upper limit pressure, and/or whether the input pressure is approximately equal to the intermediate pressure. If too much pressure is being applied, the feedback system 106′ may output feedback that informs the user to reduce pressure on the input device 108′. If too little pressure is being applied, the feedback system 106′ may output other feedback that informs the user to increase pressure on the input device 108′. Moreover, if an ideal pressure is being applied, then the feedback system 106′ may output other feedback to inform the user to maintain the current amount of pressure.

Moreover, in some embodiments, the pressure sensor 117′ may detect a pressure distribution that is associated with the tactile input. More specifically, the user may touch the input device 108′, and the pressure sensor 117′may detect how pressure is being distributed across the input device 108′ during the test. In some embodiments, a pressure map may be detected and generated by the sensor 117′. The processor 120′ may compare this pressure distribution to a predetermined pressure distribution stored in the data storage device 122′. Feedback from the feedback system 106′ may inform and train the user how to touch and distribute pressure across the input device 108′ during the impairment test.

While at least one exemplary aspect has been presented in the foregoing detailed description of the invention, it should be appreciated that a vast number of variations exist. It should also be appreciated that the exemplary aspect or exemplary aspects are only examples, and are not intended to limit the scope, applicability, or configuration of the invention in any way. Rather, the foregoing detailed description will provide those skilled in the art with a convenient road map for implementing an exemplary aspect of the invention. It being understood that various changes may be made in the function and arrangement of elements described in an exemplary aspect without departing from the scope of the invention as set forth in the appended claims.

What is claimed is:

1. An impairment detection device configured to perform an impairment analysis on a user, the impairment detection device comprising:
an input device configured to receive a user input during a testing phase of the impairment analysis;
a first sensor configured to analyze the user input to observe an amount of an impairing substance of the user;
a second sensor configured to observe a current performance parameter associated with the input, the current performance parameter being changeable by the user during the testing phase;
a feedback device configured to provide feedback to the user during the testing phase; and
a controller with a processor configured to compare the current performance parameter to a predetermined target parameter and determine a difference between the current performance parameter and the predetermined target parameter;
the controller configured to control the feedback device to provide feedback during the testing phase, the feedback configured to inform the user how to change the current performance parameter to reduce the difference between the current performance parameter and the predetermined target parameter.

2. The impairment detection device of claim 1, wherein the user input is a sample of breath delivered from the user;
wherein the first sensor is configured to analyze the sample of breath to observe an amount of an impairing substance in the sample of breath; and
wherein the second sensor is configured to observe a current flow rate at which the sample of breath is delivered from the user to the input device.

3. The impairment detection device of claim 2, wherein the processor is configured to compare the current flow rate to a predetermined target flow rate and determine a difference between the current flow rate and the predetermined target flow rate; and
wherein the feedback is configured to inform the user how to change the current flow rate to reduce the difference between the current flow rate and the predetermined target flow rate.

4. The impairment detection device of claim 2, wherein the processor is configured to compare the current flow rate to a predetermined lower limit flow rate and determine a difference between the current flow rate and the predetermined lower limit flow rate; and
wherein the feedback is configured to inform the user to increase the current flow rate to at least equal the predetermined lower limit flow rate.

5. The impairment detection device of claim 2, wherein the processor is configured to compare the current flow rate to a predetermined upper limit flow rate and determine a difference between the current flow rate and the predetermined upper limit flow rate; and
wherein the feedback is configured to inform the user to decrease the current flow rate to at most equal the predetermined upper limit flow rate.

6. The impairment detection device of claim 2, wherein the second sensor is configured to observe a current mass flow rate at which the sample of breath is delivered from the user to the input device.

7. The impairment detection device of claim 6, wherein the second sensor is a thermal mass flow meter.

8. The impairment detection device of claim 3, further comprising a third sensor configured to observe a current quantity of delivered breath;
wherein the processor is configured to compare the current quantity of delivered breath to a predetermined quantity limit;
wherein the controller is configured to control the feedback to provide a first feedback and a second feedback;
wherein the first feedback is configured to inform the user how to change the current flow rate to reduce the difference between the current flow rate and the predetermined target flow rate; and
wherein the second feedback is configured to inform the user that the current quantity of delivered breath is at least equal to the predetermined quantity limit.

9. The impairment detection device of claim 1, wherein the input device includes a housing that supports the first sensor and the second sensor; and
wherein the feedback device is remote from the housing of the input device.

10. The impairment detection device of claim 9, wherein the feedback device is supported by a vehicle.

11. The impairment detection device of claim 1, wherein the input device is configured to receive a tactile user input;
further comprising an emitter configured to emit a light proximate the input device to produce a light response associated with the tactile user input; and
wherein the first sensor is configured to analyze the light response to observe the amount of the impairing substance.

12. The impairment detection device of claim 11, wherein the second sensor is configured to observe a current pressure associated with the tactile user input;
wherein the feedback is configured to reduce the difference between the current pressure and a predetermined pressure.

13. The impairment detection device of claim 12, wherein the second sensor is configured to observe a current pressure distribution across the input device; and
wherein the feedback is configured to reduce the difference between the current pressure distribution and a predetermined pressure distribution.

14. A method of operating an impairment detection device, the impairment detection device including an input device, a controller with a processor, a first sensor, a second sensor, and a feedback system, the method comprising:
analyzing, with the first sensor, a user input provided during a testing phase to the input device;
observing, with the first sensor, an amount of an impairing substance of the user as a result of the analysis of the user input;
observing, with the second sensor, a current performance parameter associated with the user input, the current performance parameter being changeable by the user during the testing phase;
comparing, with the processor, the current performance parameter to a predetermined target parameter to obtain a difference between the current performance parameter and the predetermined target parameter; and
providing, with the feedback system, feedback configured to inform the user how to change the current performance parameter during the testing phase to reduce the difference between the current performance parameter and the predetermined target parameter.

15. The method of claim 14, wherein the user input is a sample of breath delivered from the user;
wherein analyzing the user input includes analyzing the sample of breath to observe an amount of an impairing substance in the sample of breath; and
wherein observing the current performance parameter includes observing a current flow rate at which the sample of breath is delivered from the user to the input device.

16. The method of claim 15, wherein comparing the current performance parameter to the predetermined target parameter includes comparing the current flow rate to a predetermined target flow rate;
further comprising determining a difference between the current flow rate and the predetermined target flow rate; and
further comprising providing feedback configured to inform the user how to change the current flow rate to reduce the difference between the current flow rate and the predetermined target flow rate.

17. The method of claim 15, wherein comparing the current performance parameter to the predetermined target parameter includes comparing the current flow rate to a predetermined lower limit flow rate;
further comprising determining a difference between the current flow rate and the predetermined lower limit flow rate; and
further comprising providing feedback configured to inform the user to increase the current flow rate to at least equal the predetermined lower limit flow rate.

18. The method of claim 15, wherein comparing the current performance parameter to the predetermined target parameter includes comparing the current flow rate to a predetermined upper limit flow rate;
further comprising determining a difference between the current flow rate and the predetermined upper limit flow rate; and
further comprising providing feedback configured to inform the user to decrease the current flow rate to at most equal the predetermined upper limit flow rate.

19. The method of claim 16, further comprising:
observing a current quantity of the sample;
comparing the current quantity to a predetermined quantity limit; and
providing a first feedback and a second feedback;
wherein the first feedback is configured to inform the user how to change the current flow rate to reduce the difference between the current flow rate and the predetermined target flow rate; and
wherein the second feedback is configured to inform the user that the current quantity of delivered breath is at least equal to the predetermined quantity limit.

20. The method of claim 14, wherein the user input is a tactile user input;
further comprising emitting a light toward a body part to produce a light response associated with the tactile user input;
wherein observing the amount of the impairing substance includes observing the light response to observe the amount of impairing substance;
wherein observing the current performance parameter includes observing a current pressure associated with the tactile user input; and
wherein the feedback is configured to reduce the difference between the current pressure and a predetermined pressure.
